(19) United States
(12) Patent Application Publication
(10) Pub. No.: US 2025/0127653 A1
(43) Pub. Date: Apr. 24, 2025

(54) HYPERTHERMIC TREATMENTS WITH APPLIED MAGNETIC FIELDS

(71) Applicant: Robert E Sandstrom, Longview, WA (US)

(72) Inventor: Robert E Sandstrom, Longview, WA (US)

(21) Appl. No.: 18/920,195

(22) Filed: Oct. 18, 2024

Related U.S. Application Data
(60) Provisional application No. 63/591,216, filed on Oct. 18, 2023.

(51) Int. Cl. A61F 7/00

(52) U.S. Cl. CPC A61F 7/00; A61F 2007/0052

(57) ABSTRACT

Methods of hyperthermic treatment, including heating a tissue to a temperature range of 40° C. to 45° C., and exposing the heated tissue to a magnetic field having a field strength that is capable of extending the lifetime of a free radical entity within the heated tissue. The prolonged lifetime of the free radical entity in the heated tissue enhances the therapeutic effects of the treatment, leading to improved outcomes in various medical applications.

### TECHNICAL FIELD

The present disclosure relates to therapeutic methods that include hyperthermia, and more particularly to the use of a magnetic field to prolong the lifetimes of free radical entities produced during hyperthermic treatment.

### BACKGROUND

Hyperthermia is a medical treatment which employs elevated local, regional, or general body temperature elevation to treat any of a range of clinical conditions. During hyperthermic treatment, elevated local and/or general body temperatures are created and sustained in the subject. For example, hyperthermic treatments might elevate temperatures of the tissue undergoing treatment to the region of 40° C. to 45° C.

Hyperthermic treatments result in the production of free radicals. Free radicals are chemical entities which possess a single, unpaired electron in an outer electron orbital, and are therefore subject to the effects of an applied magnetic field. In particular, free radical entities precess in a magnetic field of a specific strength. The magnetic field strength required for such precession is dictated by the quantum state of the radical within the magnetic field. Precession in the magnetic field is comparable to classical Larmor Precession, while the dynamics of free radical behavior in quantum terms is governed by the Pauli Exclusion Principle by virtue of the electron spin state of the radical.

A specific and relatively low strength magnetic field can therefore prolong free radical reactivity and limit immediate free radical recombination, increasing the efficacy of hyperthermic treatments.

### SUMMARY

The present disclosure is directed to methods of hyperthermic treatment, the methods including heating a tissue to a temperature range of 40° C. to 45° C., and exposing the heated tissue to a magnetic field having a field strength that is capable of extending the lifetime of a free radical entity within the heated tissue.

### DETAILED DESCRIPTION

Local hyperthermia can be used to treat cancers and other dermatologic conditions at skin and mucosal surfaces, with the goal of killing tumor tissue while sparing normal or healthy tissue. Regional hyperthermia may heat a larger portion of the subject, such as an entire organ or a limb, and may be used to weaken cancer cells so that they are more likely to be killed by additional treatments used in combination with hyperthermia. General hyperthermia (or whole-body hyperthermia) elevates the temperature of the entire body of the subject, and can be used where a condition affects the entire subject, such as the metastasis of a cancer.

The heating employed during hyperthermia can be generated by any suitable heating process, such as for example the application of electromagnetic radiation, such as microwave, infrared, or radio frequencies, or the application of ultrasound. Alternatively, or in addition, the desired heating of tissue can be accomplished by direct application of thermal energy, such as via the application of thermal energy via a needle or probe. If heating is being applied locally, tissues can be heated to temperatures above 40° C. to 45° C.

Hyperthermic treatment can also be accomplished by infusion of warmed liquids, such as by infusion of a subject's limbs, their peritoneal cavity, or pleural cavity, among others. Alternatively, or in addition, whole body hyperthermic treatment can include entire body infrared treatment, such as by using a hyperthermia dome, using hyperthermia wrappings on all or a portion of the subject's body, or simply elevating the temperature of the room that the subject occupies.

In some instances, hyperthermic treatment can be accomplished by the application of magnetic nanoparticles to the tissue of interest, and an application of an external magnetic field heats the magnetic nanoparticles by induction. However, the magnetic field strengths required for this type of heating are much higher than the field strengths that are appropriate for prolonging the lifetimes of free radical entities.

Typically, hyperthermic treatments include target temperature ranges and target hyperthermia durations selected based upon the clinical condition being treated, and the nature of the subject. During hyperthermia, a target tissue can be exposed to, or heated to, temperatures in the range of 40° C. to 45° C. Hyperthermic treatments may be repeated, but typically not more frequently than twice a week. Hyperthermic treatments are typically carried out based upon a defined and preselected treatment schedule.

The application or induction of hyperthermic temperatures may damage organs and tissues adjacent to the target tissue. Hyperthermia may indirectly damage the brain, lung tissue and/or internal organs of the subject. Potential complications of hyperthermia can include systemic shock, the formation of blood clots, swelling, bleeding, and infection.

Hyperthermia can be employed in treating infectious diseases, or employed in combination with one or more alternative treatment regimens, including chemotherapy and/or radiation therapy, particularly during treatment of cancers. Some current research in hyperthermic treatments include improved methods of monitoring temperatures of adjacent and/or at-risk tissues. Other current research can involve the use of nanoparticles to induce or enhance hyperthermic interventions.

The application of a magnetic field having a low field strength (10-500 Gauss) can be used to extend the lifetime of free radical entities generated during hyperthermia. This low field strength has been described as a paradoxical low field effect, and arises as symmetry breaking favors singlet state degeneracy, and favors singlet to triplet interconverson with a range of vector states that are incompatible with recombination. The singlet/triplet interconversion is not favored by magnetic field strengths that exceed the hyperfine energy levels of the radicals. The energy level seen in the Zeeman effect relates to the hyperfine levels.

By extending free radical lifetimes through the imposition of a low strength magnetic field in this way, the effectiveness and/or efficiency of hyperthermic treatments can be enhanced.

In one aspect of the present disclosure, the hyperthermic treatment is a local treatment, and the application of the magnetic field is similarly local. For example, where the hyperthermic apparatus employs an armature for positioning the source of heating over the tissue being treated, an appropriate electromagnetic generator can be attached to the same armature, so that the electromagnetic generator can be placed adjacent to, or directly over, the hyperthermic treatment site. In another aspect of the present disclosure, the hyperthermic treatment is a general treatment, involving the heating of substantially the entire subject.

The present methods are agnostic as to the source of hyperthermic heating of the tissue under treatment, provided that a magnetic field of the appropriate and specific magnetic strength is employed. In one aspect of the present disclosure, the strength of the magnetic field employed is between 0.01 and 5.0 Tesla.

The generated magnetic field can be substantially uniform in field strength across the volume of the heated tissue. Alternatively, the magnetic field can be generated so that the field strength is contoured across the volume of application. The magnetic field strength can additionally or alternatively be a static magnetic field, or a dynamic magnetic field that changes field strength over time, although a static magnetic field can offer some physiological advantages.

An engineer of ordinary background and education with regards to electromagnetic field generation devices can design and fabricate apparatus having electromagnetic coils that are capable of generating sufficiently uniform and/or contoured magnetic fields of the specific field strength, as described herein, and which would be suitable for the treatment methods described herein.

For example, an appropriate field generation device may incorporate appropriate shielding materials, such as for example employing aluminum or other magnetic insulating materials, in order to focus the generated magnetic field as while maintaining a desired a specific and uniform magnetic field having a desired three-dimensional contour. The magnetic field device can be sized to be used locally, for example to treat specific tumor sites. Alternatively, the magnetic field device can be large enough to enclose and treat entire limbs, larger portions of the subject's body, and/or the entire body of the subject.

An appropriate magnetic field device for the purposes of the present disclosure may include one or more a magnetic field generators, a magnetic field sensor, a data entry panel that permits a user to input time periods and electromagnetic field strengths for an electromagnetic field production regime, and a data processor including non-transitory computer readable memory, adapted to control said magnetic field generator to output a magnetic field in accordance with data received from said data entry panel.

The requisite magnetic field can be generated by any appropriate magnetic field source, such as for example ferromagnets, or rare earth magnetic elements, particularly for local treatments where a magnetic field of limited volume can be contained within a device attached to or employed as a heating source. For application of larger magnetic fields, the magnetic field source may include one or more Helmholtz coils.

In one aspect, the magnetic field sources are arrayed such that the magnetic field can be contoured to fit the size and shape of the subject, or area to undergo treatment. Alternatively, or in addition, one or more magnetic field sensors connected to the control station can be used to monitor the magnetic field in multiple axes. In one embodiment, the magnetic field sensors are Hall probes.

In some aspects, the methods of hyperthermic treatment disclosed herein include heating a tissue to a temperature of 40° C. to 45° C.; and exposing the heated tissue to a magnetic field having a field strength sufficient to prolong a lifetime of a free radical entity in the heated tissue.

In some aspects, the methods disclosed herein relate to methods of hyperthermic treatment wherein the tissue is or includes skin tissue, and the hyperthermic treatment is selected to treat a dermatologic condition in or on the tissue.

In some aspects, the methods disclosed herein relate to methods of hyperthermic treatment wherein the hyperthermic treatment is selected to treat an infection in the tissue.

In some aspects, the methods disclosed herein relate to methods of hyperthermic treatment wherein the hyperthermic treatment is selected to treat a cancer in the tissue.

In some aspects, the methods disclosed herein relate to methods of hyperthermic treatment wherein the hyperthermic treatment is combined with a chemotherapeutic treatment.

In some aspects, the methods disclosed herein relate to methods of hyperthermic treatment wherein the hyperthermic treatment is combined with a radiation treatment.

In some aspects, the methods disclosed herein relate to methods of hyperthermic treatment wherein the hyperthermic treatment is a local hyperthermic treatment.

In some aspects, the methods disclosed herein relate to methods of hyperthermic treatment wherein the magnetic field is created by at least one magnet positioned exterior to the heated tissue.

In some aspects, the methods disclosed herein relate to methods of hyperthermic treatment wherein exposing the heated tissue to the magnetic field includes placing a cone-shaped electromagnetic generator adjacent to the heated tissue.

In some aspects, the methods disclosed herein relate to methods of hyperthermic treatment wherein exposing the heated tissue to the magnetic field includes exposing the heated tissue to a magnetic field having a field strength of between 1-500 gauss.

In some aspects, the methods disclosed herein relate to methods of hyperthermic treatment wherein exposing the heated tissue to the magnetic field includes exposing the heated tissue to a magnetic field having a field strength of between 1-100 gauss.

In some aspects, the methods disclosed herein relate to methods of hyperthermic treatment wherein exposing the heated tissue to the magnetic field includes exposing the heated tissue to a magnetic field having a field strength of between 30-50 gauss.

In some aspects, the methods disclosed herein relate to methods of hyperthermic treatment wherein exposing the heated tissue to the magnetic field includes exposing the normal tissue to the magnetic field for between 5 minutes and 3 hours.

In some aspects, the methods disclosed herein relate to methods of hyperthermic treatment wherein exposing the heated tissue to the magnetic field includes exposing the normal tissue to the magnetic field for between 20 minutes and 2 hours.

In some aspects, the methods disclosed herein relate to methods of hyperthermic treatment wherein exposing the heated tissue to the magnetic field includes exposing the normal tissue to the magnetic field for between 40 minutes and 1.5 hours.

In the description and the claims, the term “substantially” means a deviation of up to 10% of the stated value, if physically possible, both downward and upward, otherwise only in the appropriate direction; in the case of degrees (angle and temperature), and for indications such as “parallel” or “normal,” this means ±10°. For terms such as “substantially constant” etc., what is meant is the technical possibility of deviation which the person skilled in the art proceeds from, and not the mathematical one. For example, a “substantially L-shaped cross-section” comprises two elongated surfaces, which merge at one end into the end of the other surface, and whose longitudinal extensions are arranged at an angle of 45° to 120° to each other.

All given quantities and percentages, in particular those relating to the limitation of the invention, insofar as they do not relate to specific examples, are understood to have a tolerance of ±10%; accordingly, for example: 11% means 9.9% to 12.1%. With terms such as “a solvent,” the word “a” is not to be considered to represent a singular numeral, but rather is to be considered an indefinite article or pronoun, unless the context indicates otherwise.

The term: “combination” and/or “combinations,” unless otherwise stated, mean all types of combinations, starting from two of the relevant components up to a plurality or all of such components; the term “containing” also means “comprising.”

Although the present methods and apparatus have been shown and described with reference to the foregoing operational principles and preferred embodiments, it will be apparent to those skilled in the art that various changes in form and detail may be made without departing from the spirit and scope of the invention. The present invention is intended to embrace all such alternatives, modifications and variances that fall within the scope of the appended claims.

Inventions embodied in various combinations and subcombinations of features, functions, elements, and/or properties may be claimed through presentation of new claims in related applications. Such new claims, whether they are directed to a different invention or directed to the same invention, whether different, broader, narrower or equal in scope to the original claims, are also regarded as included within the subject matter of the present disclosure.

What is claimed is:

1. A method of hyperthermic treatment, comprising:
heating a tissue to a temperature of 40° C. to 45° C.; and
exposing the heated tissue to a magnetic field having a field strength of 1-500 gauss in order to prolong a lifetime of a free radical entity in the heated tissue.

2. The method of claim 1, wherein the tissue is or includes skin tissue, and the hyperthermic treatment is selected to treat a dermatologic condition in or on the tissue.

3. The method of claim 1, wherein the hyperthermic treatment is selected to treat an infection in the tissue.

4. The method of claim 1, wherein the hyperthermic treatment is selected to treat a cancer in the tissue.

5. The method of claim 1, wherein the hyperthermic treatment is combined with a chemotherapeutic treatment.

6. The method of claim 1, wherein the hyperthermic treatment is combined with a radiation treatment.

7. The method of claim 1, wherein the hyperthermic treatment is a local hyperthermic treatment.

8. The method of claim 1, wherein the magnetic field is created by at least one magnet positioned exterior to the heated tissue.

9. The method of claim 1, wherein exposing the heated tissue to the magnetic field includes placing a cone-shaped electromagnetic generator adjacent to the heated tissue.

10. The method of claim 1, wherein exposing the heated tissue to the magnetic field includes exposing the heated tissue to a magnetic field having a field strength of between 1-100 gauss.

11. The method of claim 1, wherein exposing the heated tissue to the magnetic field includes exposing the heated tissue to a magnetic field having a field strength of between 30-50 gauss.

12. . . . The method of claim 1, wherein exposing the heated tissue to the magnetic field includes exposing the normal tissue to the magnetic field for between 5 minutes and 3 hours.

13. The method of claim 1, wherein exposing the heated tissue to the magnetic field includes exposing the normal tissue to the magnetic field for between 20 minutes and 2 hours.

14. The method of claim 1, wherein exposing the heated tissue to the magnetic field includes exposing the normal tissue to the magnetic field for between 40 minutes and 1.5 hours.
